(19) United States
(12) Patent Application Publication
(10) Pub. No.: US 2021/0236684 A1
(43) Pub. Date: Aug. 5, 2021

(54) DEVICE FOR MAPPING THE SHAPE OF A SPATIAL FORM

(71) Applicant: Exal Bone SP ZO.O., Jasionow (PL)

(72) Inventors: Mateusz SIWAK, Lodz (PL); Lukasz PIOTROWSKI, Lodz (PL)

(21) Appl. No.: 17/049,107

(22) PCT Filed: Apr. 20, 2019
(86) PCT No.: PCT/IB2019/053282

§ 371 Date: Oct. 20, 2020

(30) Foreign Application Priority Data
Apr. 20, 2018 (PL) PL-425300

(51) Int. Cl. A61L 15/14; A61L 15/12; C08K 3/22; C09J 183/04; C08K 5/00

(52) U.S. Cl. CPC A61L 15/14; A61L 15/125; C08K 2003/2275; C09J 183/04; C08K 5/0016; C08K 3/22

(57) ABSTRACT

The apparatus for mapping the shape of the spatial form according to the invention comprises a thermoplastic sheet provided with a system adapted to generate heat capable of plasticizing said sheet under the influence of the current flowing through said system. The subject of the invention is also a device for immobilizing human or animal body parts, in particular limbs or joints, the immobilizing device comprising said device for mapping the shape of the spatial form. The subject of the invention is also a system comprising an apparatus for mapping the shape of a spatial form.

The subject of the invention is a device for mapping the shape of a spatial form comprising a thermoplastic sheet provided with a system adapted to generate heat and capable of plasticizing said sheet due to flow of the current through said system. The subject of the invention is also a device for immobilizing human or animal body parts, in particular limbs or joints, the immobilizing device comprising said device for mapping the shape of the spatial form. The subject of the invention is also a system comprising the device for mapping the shape of a spatial form.

Mapping the shape of the spatial form is needed to prepare a replica of a spatial structure such as a sculpture or foot in order to make a model for making shoes on order. Mapping the shape of the spatial form is also used for the production of medical devices in the form of devices for immobilizing parts of the body which are used in medicine in the treatment of e.g. bone fractures or correction of their mutual position within the joint in the case of congenital clubfoot treatment. It is essential that physiological form of the device is obtained in order to immobilize the limb, limbs and/or joint, as it allows proper fusion of bones or their reposition relative to each other, so that they are not exposed to external factors that can disturb the treatment process, and also force physiological position of the immobilized limb. It is important that such devices have the smallest weight possible, which will not be an excessive burden for the patient.

In the state of the art there are known devices for mapping the shape of a spatial form. Preparation of a mold corresponding to the shape of the spatial form is required in particular to obtain immobilizing devices that are used to impart the desired position of the supported part of the body or to immobilize the supported portion relative to other parts of the body.

Numerous immobilizing devices have been presented in the patent literature, among others castings, rails, braces and stiffening devices. Traditionally, casting based on bandages containing anhydrous gypsum (CaSO₄) have been used for making them, which was justified by the low price of such a solution. Due to the high weight of materials for the preparation of such stiffening dressings, the inability to clean them easily, as well as removal, gypsum over the time has been replaced with synthetic casting materials (such as fiberglass soaked in polyurethane resin). Such materials are lighter and can be cleaned, but have a rough outer surface and are still relatively heavy and bulky. In addition, in contrast to gypsum stiffening plaster, the products used to manufacture synthetic materials are more resistant to stress, more difficult to break and do not crumble, which in turn leads to a much higher durability of such products, which in the case of traditional casts is around 2 weeks. It is worth mentioning here that in the process of stiffening gypsum plaster, heat is generated, which poses a real risk of burns. It should also be noted that during a relatively short time of forming and stiffening the dressing with the use of synthetic materials, it is often not possible to properly shape the form to ensure proper fracture healing, while the edges of the obtained form often remain sharp and can cause injuries.

Both in the case of casting based on traditional bandages containing gypsum and those obtained using plastics, in order to disassemble the device it is necessary to use special oscillating saws, the use of which creates the risk of injury.

Thermoplastic materials such as those described in U.S. Pat. No. 4,240,415 are currently used to form castings of orthodontic appliances and other immobilizing devices. These thermoplastic materials are produced in the form of extruded sheets, which when brought to a softening point (50°0 C. to 100° C.) can be formed and adapted to fit and form a precise mold around a body part such as a limb, said molding possibility is available as long as the material does not stiffen. These materials can also be re-heated and restored to the original shape, after which they can then be formed into a new shape. The preparation of such a sheet, consisting primarily of its plasticization, takes place in external heating devices that require additional space in the office wherein the stiffening dressing is applied. The plasticization of the stiff plastic also requires removing the sheet from the patient's body every time and re-placing it in the external heating device. The necessity of repeated removal and putting on a stiffening dressing (for proper fitting) sometimes causes additional pain in the patient, and in the case of fresh fractures, it may result in a secondary reposition of already focused fragments. U.S. Pat. No. 4,060,075 describes a rail system that is formed of a transformable material embedded in a fabric that may include a fastener, such as a zip fastener or hook and loop material (VELCRO). In this rail system, the two-component plastic is mixed and formed into a double-walled sheet, which is then installed around the body part before the plastic mixture hardens. It is difficult to obtain an even casting thickness and a casting surface with this rail system, and when the cast hardens, it is very stiff and does not show elasticity. In addition, the casting can not be re-used and can not be perforated to provide ventilation. It is also problematic to properly fit the device to the circumference of the limb (different patients have different anthropometric dimensions of the limbs). In addition, the limb can be deformed due to transient swelling. The dressing can not compress too much, because it can cause swelling, limb ischemia, which can lead to its amputation. When the swelling disappears, the dressing may be too loose, so it will be necessary to replace it with a new one.

European patent application EP 401 883 describes a thermoplastic immobilizing device made of an extruded thermoplastic material that is surrounded by a fabric. The device is equipped with a zip fastening connecting two opposite edges of a cast or splint, thanks to which it was possible to adjust it more precisely to the shape of the limb, which, as indicated above, may change due to the occurrence and disappearance of swelling. An embodiment of the device according to patent EP 401 883 consists of a thermoplastic material in a plain type fabric with a zipper. The device is vacuum-packed in plastic to prevent the material from getting wet while melting the thermoplastic material. The vacuum-packed device is placed in water at the temperature required to soften the thermoplastic material for forming. After opening the plastic packaging, the casting can not be processed without watering the canvas or the risk of burning the fabric with a heat gun. A flexible textile or “stockinette” material tends to detach from thermoplastic material and swell in areas that are stretched and formed, which causes potential problems with the pressure of the patient when the cast is in place. In addition, the thermoplastic material is very sticky after softening, making molding difficult.

As indicated above, the available solutions do not offer a device shape of which can be formed without limiting the stiffening time of the stiffening cast and they do not allow correction of the limb position after stiffening of the cast.

It is advisable to develop a new solution that allows to obtain a device that would be safe for the user, durable and resistant to damage, could be formed without limitation related to the time of stiffening of the material from which it was made, so that it would be possible to obtain a suitable fit for each application. In addition, if the solution is used as a fixation device for the patient's body parts, it is expected that the position of the immobilized parts of the body can be corrected after stiffening the sheet from which the immobilizing device has been made, and it is desirable that said correction could take place without destroying said sheet at the same time, e.g. by cutting it.

These objectives have been achieved by providing a solution as defined by independent claims 1, 18, 19. Favorable variants of the solution are defined by dependent claims.

The apparatus for mapping the shape of the spatial form comprising a thermoplastic sheet according to the invention is characterized in that the thermoplastic sheet is provided with a flexible system for generating heat to plasticize said sheet due to flow of the current. Preferably, the thermoplastic system is provided with means for connecting the current.

Preferably, the system for generating heat is provided in the form of a conductor (s) system arranged in a thermoplastic sheet in the form of a grid, a sinusoid, a spiral or a broken strip. Preferably, said conductor is also provided with insulation suitable for the voltage to be applied.

Preferably, the thermoplastic sheet consists of a top layer and a base layer, whereby on the surface of the base layer facing the top layer grooves are provided to receive the conductor laid in said layer. Advantageously also, on the surface of the top layer facing the base layer projections are provided having size and shape substantially corresponding to the size and shape of the channels in the base layer, the height of said projections being smaller than the depth of the grooves, and the difference between said depth and said height essentially corresponds to the height of the conductor laid in the base layer. Preferably, said top and base layers are joined by a flexible and thermally conductive binder, preferably the binder being a universal silicone. Preferably, according to the invention, said layers are made by injection molding either using a 3D printer or using stamped molds.

Preferably, perforation is provided in the areas of the sheet defined between the conductors forming the heat generating system in the sheet.

Preferably, the thermoplastic sheet is made of a material selected from thermoplastic polymers, in particular: thermoplastic elastomers such as, among others: thermoplastic polyurethanes, thermoplastic polyisoprenes, thermoplastic polyesters, thermoplastic polyolefins, polyvinylchloride, polystyrene, blends of two or more of these materials. It is also preferred that the thermoplastic sheet is made of thermoplastic polymers selected from the group consisting of thermoplastic polyurethane, isotactic polypropylene, ethylene-1-butene copolymers, ethylene 1-ethylene copolymer, poly-e-caprolactone, ε-polycaprolactone thermoplastic polyurethane or a blend of two or more from these materials. Preferably, the thermoplastic sheet is made of a blend based on polycaprolactone with the addition of plasticizers.

Preferably, the thermoplastic sheet is made of a material having a softening point in the range of 38 to 100 degrees Celsius.

Preferably, the device according to the invention comprises means for connecting, preferably releasably connecting, the opposite edges of the thermoplastic sheet.

Also preferably the device according to the invention comprises a heat-insulating layer on the surface of a thermoplastic sheet.

The subject of the invention is also a device for immobilizing a human or animal body part, in particular a limb or a joint, characterized in that said device comprises a device for mapping the shape of the spatial form according to the invention.

The subject of the invention is also a system comprising device for mapping the shape of a spatial form according to the invention and a controller controlling the system parameters.

### DETAILED DESCRIPTION OF THE INVENTION

The mapping of the shape of the spatial form in the sense of the invention should be understood as a negative representation of the shape of said form, such as a casting mold, a plaster dressing or orthopedic scales.

The thermoplastic sheet in the device for mapping of the shape of the spatial form according to the invention means a flat sheet having a thickness in the range of about 1.5 mm to about 3.5 mm. made of thermoplastic material. Preferably the sheet thickness is about 3 mm. Materials with thermoplastic properties are known to the person skilled in the art. Examples of preferred materials for making a thermoplastic sheet include materials selected from thermoplastic polymers, in particular: thermoplastic elastomers such as thermoplastic polyurethanes, thermoplastic polyisoprenes, thermoplastic polyesters, thermoplastic polyolefins, polyvinylchloride, polystyrene, blends of two or more listed materials. Other preferred examples of thermoplastic materials include thermoplastic polyolefins selected from the group consisting of thermoplastic polyurethane, isotactic polypropylene, copolymers of ethylene with 1-butene, ethylene copolymer with 1-ethene, poly-e-caprolactone, polyepolactone-containing polyurethane containing ε-polycaprolactone or a blend of two or more of these materials. A particularly preferred example of a material that can be used to make a thermoplastic sheet is a polycaprolactam-based blend with the addition of plasticizers (e.g., CoolMorph Plastic™ from Thermoworx Ltd).

The flexible heat generation system with which the thermoplastic sheet in the device for mapping of the shape of the spatial form according to the invention is provided means a system of current-conductive elements placed inside the sheet and capable of generating heat due to the flow of the current, whereby the current flow as a result of which the heat is generated can be caused by the influence of electromagnetic induction or by connecting the system for generating heat to an external circuit providing a source of power. In the second case, the system is equipped with means for connecting the external power circuit for providing the power source. Where the generation of heat is caused by the effects of electromagnetic induction, the system adapted to generate heat can be made of polymers such as oligo dimethacrylates (ε-caprolactone) enriched with superparamagnetic magnetite nanoparticles (Fe₃O₄, d=11 nm), which are capable of induction heat, i.e. capable of transforming electromagnetic energy into heat under the influence of an external high frequency field. One skilled in the art knows how to prepare a material with such desirable parameters (Electromagnetic Activation of Shape Memory Polymer Networks Containing Magnetic Nanoparticles. (2006). Macromolecular Rapid Communications, 27 (14), pp. 1168-1172.) The thermoplastic sheet prepared in this way can be heated with commercially available large-size induction heaters. During the heating stage, the temperature of the system is preferably controlled.

Under the influence of the induced eddy currents, heat is generated leading to the plastification of the material from which the sheet was made.

An important feature of the device is the fact that the system adapted to generate heat ensures obtaining a significantly lower temperature on the external surface of the device as comperted to temperature of plasticity/softening of the thermoplastic material. This feature is important because the softening temperature of many thermoplastic materials is higher than the temperature tolerated by human skin (temperature 45 degrees Celsius is considered in humans as the physiological threshold of pain).

There is known in the prior art a method for the synthesis of biodegradable, thermoplastic polymers (having shape memory) composed of photoselective oligo (ε-caprolactone) dimethacrylates and butyl acrylate (BA) as a comonomer. in these materials, the temperature of plasticity depends on the melting of the crystallizable segments of oligo (e-caprolactone) and occurs between 43 and 49° C. Parallel to this activity and independently of it, the development of segmented shape memory is obtained. Also known are polyurethanes containing magnetic nanoparticles.

A key feature of the system adapted to generate heat is also its flexibility which is appropriate for a given application and thanks to which it is possible to properly shape the sheet. Therefore, when designing the layout of conductive elements, the flexibility of the conductor should be taken into account. For example, good flexibility is provided by, for example, commercially available conductors in the form of a Teflon™ coated heating cable with a thickness. 0.7 mm (e.g. from INTO, Strzelin) placed in a 3 mm thick thermoplastic sheet made by 3D printing using for example PCL filament 1.75 mm (PCL 99 FILAMENT 750 GRAM 1.75 MM from 3D4MAKERS™. The mentioned materials are indicated by way of example, ensuring that the thermoplastic sheet equipped with conductive elements is sufficiently flexible does not extend beyond the routine activities of a person skilled in the art.

In a preferred variant of the device for mapping of the shape of the spatial form according to the invention it is provided that the heat generation system is formed of a conductor having an insulation suitable for the applied voltage. Thanks to this, it is possible to use a current with a voltage ranging from 0.5 to 240 V. The selection of a proper insulation depending on the voltage applied and the expected thermal effect is within the scope of the routine activity of a person skilled in the art.

In a preferred embodiment of the invention, the flexible heat generation system is provided as a circuit or circuits of a conductor allowing the passage of electric current, wherein the conductor is arranged inside a thermoplastic sheet in the shape of a spiral, a broken strip or a sinusoid. The conductor circuit may also be in the form of a mesh composed of a plurality of conductors arranged in parallel and perpendicular orientation relative to each other. Particularly advantageous is serial placement of the conductors arranged in parallel, thus providing heating filaments, connected at the ends with a conductor that does not make a significant contribution to the heat generated by the system. Alternatively, it is also possible a solution wherein a plurality of conductors in the form of straight wires is arranged in parallel relative one to another, which are connected to the current by circuit connecting means which are not part of the sheet. In order to limit thermal losses on the perimeter of the sheet with any conductor arrangement, it is possible to use heat insulators on the edges of the device which are applied during the time of preparation and assembly of the sheet.

In one preferred embodiment of the invention, the thermoplastic sheet consists of two layers, a top layer and a base layer, whereby on the surface of the base layer, facing the top layer, provided are grooves to receive the conductor laid in said layer. The width and depth of said grooves are provided in a size that allows the groove to accommodate the conductor arranged in the groove in such a way that it does not protrude above the plane of the cover layer, which ensures tight adhesion of the layers after application and joining of the top layer to the base layer. Preferably, a flexible and thermally conductive adhesive is used to bond the layers, in a preferred embodiment the binder is a silicone layer, e.g. a universal silicone layer.

In another preferred embodiment of the invention, in which the sheet also consists of two layers, i.e. a top layer and a base layer, projections in a size and shape substantially corresponding to the size and shape of the grooves in the base layer are provided on the surface of the cover layer, wherein the height of the mentioned projections is designed to be smaller than the depth of the grooves, and the difference between the depth of the groove and the height of the projections essentially corresponds to the size of the conductor laid in the grooves of the base layer. In case where the conductor laid in the grooves has a circular cross-section, the height of the conductor means its diameter. The case of the guide with a rectangular section, the height of the conductor will be a dimension that, when laid in the groove, runs in the axis of the dimension of the groove depth. And accordingly, in the case of laying an elliptical conductor, the height of the conductor should also be understood as its dimension running in the axis of the groove depth dimension. In case of using a conductor circuit in form of a mesh, the difference between the groove depth and the projection height essentially corresponds to twice the height of a single conductor. In this way, it is ensured that at the nodal points in the conductor circuit, the conductor does not protrude above the plane of the base layer. During the application of the surface layer to the undercoat with the conductor laid in the grooves, the projections are inserted into the grooves, which ensures a tight connection of the two layers, and the connection is additionally reinforced with a conductive adhesive placed between the layers, whereby in a preferred embodiment said adhesive is a silicone layer, e.g. universal silicone layer. The coupling of both layers is ensured by welding the thermoplastic material under load.

Said thermoplastic sheet layers are made in a known manner using technologies such as e.g. 3D printing, injection molding or press molding, e.g. from raw material in the form of granules.

It is clear to the person skilled in the art, that other methods are also available to provide a flexible system for generating heat within the thermoplastic material, thereby forming a thermoplastic sheet with a heat generating system, e.g. in the form of a mesh. Such a system can be provided by weaving conductor in the insulation, wherein the insulation is made by replacing the known insulating material granulate (such as for instance Teflon™ insulation) with a granulate of thermoplastic material, such as e.g. the previously mentioned PCL. In this way, it is possible to provide a mesh including longitudinal and transverse filaments additionally connected at the nodal points with an elastic binder or mechanical fastening. It is also possible to manufacture the system by placing the conductor system for generating heat between two continuous sheets of thermoplastic material and then gluing them with an elastic binder in the marginal parts. Then, by increasing the temperature of the system using, for example, a gas torch followed by pressing, it is possible to weld the system. In a preferred embodiment, in the thus obtained continuous thermoplastic sheet equipped with a heat generating system, in the areas lying between the conductors forming said heat generating system, a perforation can be mechanically cut out, ensuring adequate distance of the conductor from the edge of the target mesh. It is also possible to produce a sheet by placing a sheet of perforated film made of a conductor (e.g. of aluminium) between two continuous sheets of thermoplastic material, wherein said perforated sheet in this embodiment functions as the above-mentioned conductor mesh. The conductor system is further equipped with power cords and welded under pressure by connecting a current of appropriate parameters to ensure the coupling of the sheet layers.

In order to ensure the proper resistance for deformation of the hardened plastic, the thickness of the thermoplastic sheet comprising the heat generating system according to the invention is preferably between 1.5 mm and 3.5 mm. Preferably, the thickness is usually about 3 mm.

Preferably, the thermoplastic sheet may comprise a perforation, so that the desired ventilation is ensured, i.e. the air flow between the inner and outer surfaces of the sheet. Such a solution is particularly advantageous when using the device on parts of the human or animal body, In this case, i.e. in the case of using the device according to the invention to provide a device for immobilizing a human or animal part of the sheet, a perforation is preferably provided in the areas of the sheet defined between the conductors of the heat generating system, which perforation provides for an air flow between the patient's skin and the outer surface of the sheet. In a preferred embodiment, the device according to the invention may be in the form of a mesh, the mesh being a net formed of conductors forming a system for generating heat surrounded by a layer of thermoplastic material. Such an embodiment is particularly advantageous both because of the very good ability of the device to mimic the shape of the body parts of the patient, but also provides lightness and excellent ventilation.

Preferably, the thermoplastic sheet is provided from a rigid, i.e. non-plastic material at temperatures below 38 degrees Celsius and showing plasticity above said temperature, selected from thermoplastic polymers, in particular: thermoplastic elastomers such as: thermoplastic polyurethanes, thermoplastic polyisoprenes, thermoplastic polyesters, thermoplastic polyolefins, polyvinyl chloride, polystyrene blends of two or more of these materials.

In preferred embodiments, the thermoplastic sheet is made on the basis of thermoplastic polymers selected from the group consisting of thermoplastic polyurethane, isotactic polypropylene, ethylene 1-butene copolymers, ethylene 1-ethylene copolymer, poly-e-caprolactone, polyepolactone polyurethane containing ε-polycaprolactone or a blend of two or more of these materials. Preferably also the thermoplastic material may be a mixture of ε-polycaprolactone or its derivatives and another thermoplastic material. In another preferred embodiment, provision is made to use a composite material for producing a fixation element, the composite material being made of a material comprising a thermoplastic polymer containing carbon nanotubes as a fibrous reinforcement material (as in US2014052037 (A1)—2014 Feb. 20; SHEETLIKE CARBON NANOTUBE-POLYMER COMPOSITE MATERIAL).

In another preferred embodiment, the composite material is provided as a material comprising a thermoplastic polymer comprising other reinforcement material.

Preferably, the sheet is made of a material that plasticizes and allows forming at a temperature in the range of 38 to 100 degrees Celsius, which is particularly advantageous in the case of solutions for use on human or animal body parts.

In preferred embodiments of the invention, the sheet is made of polymers that melt or soften at temperatures in the range of 38° C. to 100° C., including poly (ethylene adipate), poly (epsilon-caprolactone), polyvinyl stearate, cellulose acetate, butyrate. and ethylcellulose comonomers containing poly (propylene oxide), transpolysoprene and thermoplastic materials based on polyisoprene and a polycaprolactone-based material, including commercially available polycaprolactone thermoplastic materials known as AQUAPLAST™, SYNERGY™, EZEFORM™, Coolmorph Plastic™, POLYFORM™ and POLYFLEX II™ (Smith & Nephew Roylan Inc., US).

In preferred embodiments, the thermoplastic sheet comprises means for connecting the opposing edges of the thermoplastic sheet. Such a solution makes it possible to precisely arrange the device on the surface of the spatial form, and by using fixing means allowing connection of the opposite edges of the sheet, a sleeve is formed around the spatial form, which can be precisely adapted to the spatial form.

To connect the opposite edges of the sheet, it is also possible to use the excess sheet remaining after applying portions of the sheet around the basic shape of the spatial form. By arranging the excess on both sides in the form of a flat linen seam, the excess can be used to connect the edge of the sheet to provide the sleeve. This type of connection is particularly advantageous because it allows for a perfect fit to the spatial form while maintaining very strong fastening and high aesthetic quality.

The connection of opposite edges of the sheet can preferably be provided as detachable. It is then possible to repeatedly use the same sheet, possible is its removal and re-use, e.g. after controlling the healing process. For this purpose, it is possible to use means such as, for example, a connection based on hook and loop system (VELCRO) located on the opposite surfaces of the connecting part in the form of interconnecting strips. For the same purpose, it is also possible to use latches and other means for detachably connecting said elements.

Due to the reversible plastification of the material being the basic building material of the thermoplastic sheet, the solution according to the invention can be repeatedly regenerated and adapted according to current needs.

Preferably, on one of the surfaces and between the thermoplastic sheet and the surface of the spatial form to be mapped, the sheet may comprise a heat insulating layer. As a result, the negative effect of the elevated temperature to which the thermoplastic sheet is brought can be reduced, which is of particular importance when using thermoplastic materials with a high softening temperature. Optionally, the heat-insulating layer may be attached to the surface of the sheet.

The solution containing the thermal insulation layer is particularly useful when using the device to immobilize a human or animal body part, in particular a limb, limbs or a joint. Optionally, the heat-insulating layer between the sheet and the body part may be present in the form of a garment element such as e.g. a glove or a sock. It is also possible to use a thermo insulator in the form of, e.g. a textile fabric such as jersey. This fabric may be used during the shape-setting in the device according to the invention and further protect the patient from allergic reactions to the plasticized material. In addition, the fabric can protect the limbs while forming the immobilizing device and absorb sweat, as well as prevent allergic reactions that may occur when using the device without additional protective layers. The fabric can be removed after stiffening the thermoplastic sheet.

In a particularly preferred embodiment of the invention, the device according to the invention is used to immobilize a human or animal body part, in particular a limb or limbs. The spatial form mapping device according to the invention can be used as a device for immobilizing human or animal body parts, e.g. during the treatment of bone fractures and deformities, for stabilization after sprains and joint deformities, as well as in cases of arthritis, tendonitis and cumulative trauma syndromes.

The device according to the invention can also be used for a wide range of casts, rails and orthodontic appliances, including wrist splints, cervical collars, lumbosacral restraints, supports for upper and lower limbs, chest supports, immobilizers of the knee joint, ankle braces. Lack of restrictions related to the shape of the device, and thus the established spatial form, allows for an unlimited use of the device to reproduce the shape of the upper and lower limbs, spinal segments and veterinary patients.

The device according to the invention can be part of a system, which also includes a controller controlling the parameters of the system for generating heat. The use of the controller together with the temperature sensor system enables programming of the set for different operating modes using different parameters of the current, e.g. such as fast heating of the device during preparation for its use, and slow heating modes to be used when determining the target shape to be achieved or to reposition elements of the previously fixed form. This possibility is particularly useful when using the device as an immobilizing device in situations where periodic correction of the position or rehabilitation with a fixed immobilizing device is required. The expert will have no problem with indicating the proper current parameters to achieve the desired effect, and depending on the specificity of the particular device. Preferably, the system uses current parameters from about 1 A to over 8 A.

The mentioned controller may have a manual or automatic switch or be disconnected after work. Preferably, the controller also has temperature sensors to measure the temperature of the system and the temperature at the skin, thanks to which the control over the dressing is improved, e.g. heating or switching off the heating function is ensured in order to ensure safe and comfortable use.

The mentioned is not a complete list of possible applications, therefore it can not be interpreted as limiting the use of products according to the invention.

An additional advantage of the device according to the invention is the fact that the device makes it possible to adjust the fracture with the already applied dressing and, if necessary, repositioning of incorrectly oriented bone fragments, as well as conducting some rehabilitation treatments without the necessity to remove the dressings (plasticizing for the time of the treatment, especially useful in children's orthopedics, e.g. in cases of congenital clubfoot).

The device according to the invention provides an aesthetic, light, non-itching, waterproof dressing with a wide range of applications, including in therapy and rehabilitation in the field of orthopedics.

Device for mapping the shape of a spatial form according to the invention is shown in the embodiments on the drawing, in which:

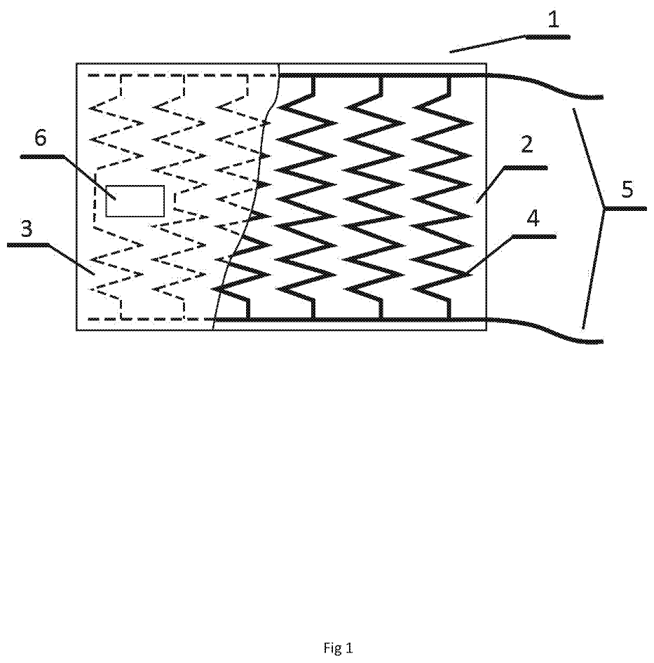

FIG. 1 schematically illustrates a top view a device for mapping the shape of a spatial form in an embodiment comprising two layers of a thermoplastic sheet and a system for generating heat, with the topsheet removed in part.

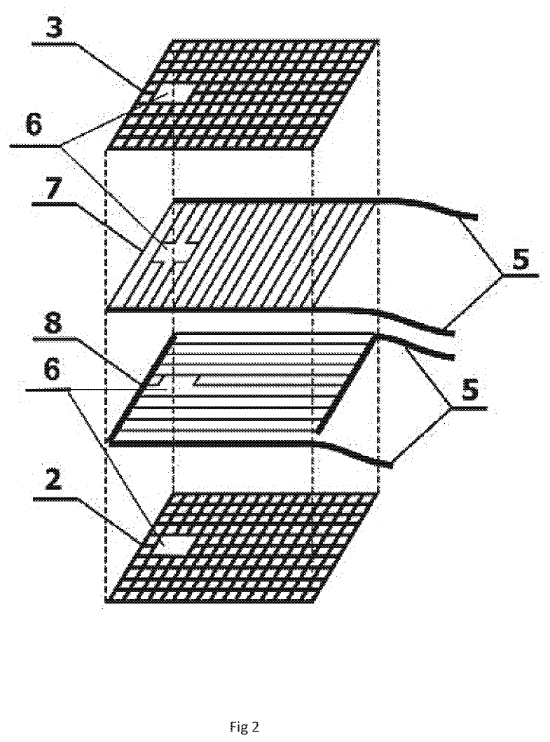

FIG. 2 schematically shows, in an exploded view, device for mapping the shape of a spatial form in an embodiment comprising two layers of thermoplastic sheet and a system for generating heat.

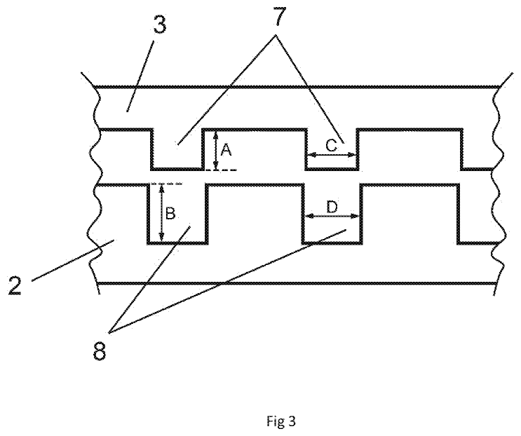

FIG. 3 is a cross-sectional view of a top layer and a base layer equipped with projections and grooves, respectively.

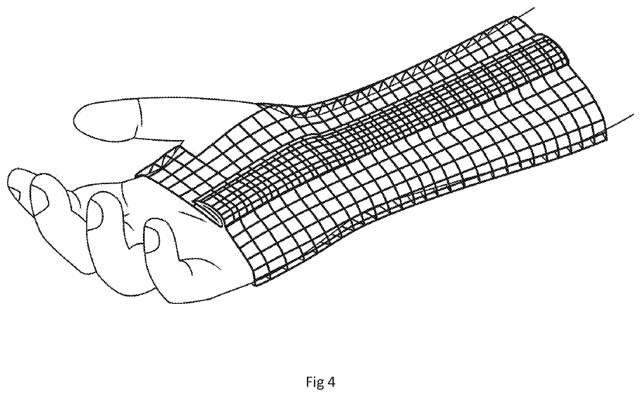

FIG. 4 shows in an embodiment a device for mapping the shape of a spatial form used as a device for immobilizing a human upper limb.

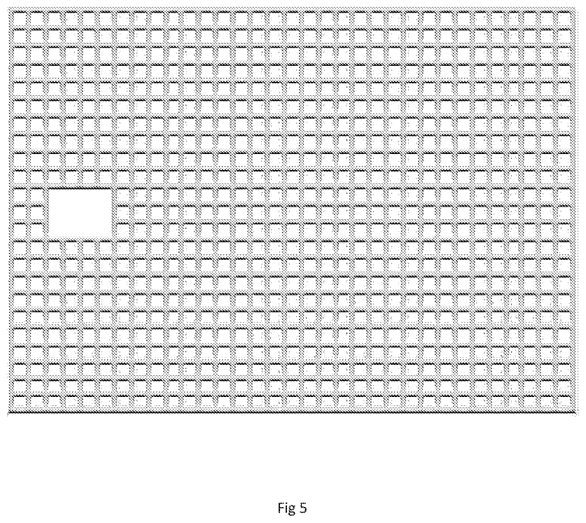

FIG. 5 shows a device for device for mapping the shape of a spatial form according to FIG. 4 in a folded-out form.

The same reference numbers in the various figures refer to the same parts of the device.

The embodiment shown in FIG. 1 illustrates a device for mapping the shape of a spatial form 1 comprising a base layer 2 and a top layer 3 together forming a thermoplastic sheet, wherein under the top layer 3 and on the base layer 2 there is provided a system for generating heat 4, equipped with means for connecting a current 5. The top layer and base layer together with the arranged system for generating heat can be combined with the heat conducting adhesive (not shown in the figure). Universal silicone may be such a binder. The top and base layers together with the arranged system for generating heat can also be placed in the area of the alternating electromagnetic field. The induced eddy currents will heat up the conductor and weld the system, providing a functional blank that can be used in the example below.

The coupling of the top and base layers can also be achieved by welding the system under pressure through connecting the system to generate heat to the source of electric current.

The conductor wires in the embodiment shown in FIG. 1 are arranged in the shape of a broken strip (zigzag). As described above, it is possible to arrange the conductor in other configurations, e.g. in the shape of a spiral, a sinusoid or a mesh.

In the embodiment shown in FIG. 1, an opening 6 is also provided. The opening 6 can be provided for an embodiment of a device for mapping the shape of a spatial form 1 used as a device for immobilizing the anterior limb of the human and is provided for receiving the thumb of a forelimb of the patient.

In FIG. 2, in an exploded view, the internal structure of the device is schematically shown in an embodiment of the invention including the base layer 2 and the top layer 3 of the thermoplastic sheet and conductor systems 7 and 8 disposed between the layers, wherein said conductor systems together form a system for generating heat in the thermoplastic sheet composed of layers 2 and 3. In the embodiment shown in FIG. 2, an opening 6 is provided in both sheet layers and the conductor systems 7 and 8. All elements of the device are placed on each as shown by the dashed line and joined together with a conductive adhesive in the form of a universal silicone, and then welded under load. In the embodiment shown in FIG. 2, the device according to the invention, after being assembled, presents itself in the form of a mesh constituted by a system for generating heat placed between layers of a thermoplastic material forming together a sheet of thermoplastic material. As can be seen in the drawing, after assembling the device elements shown in FIG. 2, no thermoplastic material is provided in the areas of the sheet defined between the conductors forming the heat generating system in the sheet. Such an embodiment is particularly advantageous both because of the very good ability of the device to reproduce the shape of the patient's body parts, but also its lightness and excellent ventilation. FIG. 4 schematically shows the use of such a device as a device for immobilizing a human front limb.

FIG. 3 schematically shows an embodiment of the base layer 2 and the top layer 3, wherein the base layer 2 has been provided with grooves 8, provided for receiving a conductor of the system for generating heat (not shown in the figure), while the top layer is provided with projections 7, which during assembly of the device are intended to fit into the grooves 8. The height A of the said projections 7 is smaller than the depth B of the grooves 8. Such configuration makes it possible to provide free space in the grooves for guiding the conductor. Typically, the height A is provided to be about 0.5 mm, while the height B is about 2 mm. In this way, a free space is provided, allowing for example for laying a conductor with a diameter of up to 1.5 mm, or in the case of a system for generating heat in the form of a mesh, it is possible to arrange a conductor with a diameter of 0.7 mm, where at the points where the wires are laid one above the other do not exceed the height of the free space defined by the difference between the depth of the grooves and the height of the projections, which in the described example is 1.5 mm.

The width C of the projections 7 is slightly smaller than the width D of the grooves 8, so that the projections 7 after applying the top layer 3 to the base layer 2 fit and fasten in the grooves 8. C may for example be 0.7 mm, D respectively may be 0.8 mm.

As indicated above, the difference between the depth B of the grooves and the height A of the projections essentially corresponds to the size of the conductor laid in the grooves of the base layer, which after joining the two layers 2 and 3 with the conductor laid in the grooves ensures tight adhesion of the layers.

FIG. 5 shows an exploded device for mapping the shape of a spatial form shown on the patient's limb in FIG. 4. In the examples, the figures do not show means for connecting the current and the controller for controlling the operating parameters of the device.

As mentioned above, a preferred embodiment of the solution can be provided, in which ferromagnetic particles are provided in the thermoplastic material. It is also possible to emboss a sheet of thermoplastic material enriched with superparamagnetic magnetite nanoparticles (Fe3O4, d=11 nm) produced in a manner known to one skilled in the art. The sheet can then be combined with a layer of thermal insulation material that protects the patient's skin surface, for example in the form of a 1 mm thick polyurethane foam layer. Due to the external source of the changing magnetic field, induced by induction coils in the form of a spiral controlled by a system dedicated for induction heaters, eddy currents are induced in the device, as a result of which heat is generated which leads to the plastification of the sheet. The external induction source may additionally include a system that optically measures the temperature of the thermoplastic sheet and a control module that regulates the operation of the system based on pyrometer readings.

After obtaining the plasticity of the sheet by generating heat plasticizing the sheet due to the current flowing in it, the device goes into the shape mapping mode. The power supply is then disconnected (or moved away from the magnetic field), and the device is formed to correspond to the shape of the spatial form. At the disappearance of the plasticity again the heat generating system is activated (or the device is placed again within the variable electromagnetic field) until the re-plasticizing of the dressing.

The layers of the thermoplastic sheet of the device according to the invention can be made using polycaprolactone, available under the trade name PCI 99 FILAMENT, and the conductor systems can be provided in the form of a 0.7 mm Teflon™ heating cable. In the embodiment where thermoplastic sheet is made by 3D printing using for example PCL filament 1.75 mm (PCL 99 FILAMENT 750 GRAM 1.75 MM from 3D4MAKERS™, the thermoplastic material has a softening temperature of about 60 degrees Celsius, while the temperature on the outer surface measured using a pyrometer is 42-43 degrees C., which is the temperature within the range of values well-tolerated by human skin.

The resulting sheet may in a preferred embodiment have a thickness of 3 mm and form a net with nodal points spaced about 1 cm apart. In a preferred embodiment of the device according to the invention, the perforation between the filaments of the net is provided in the form of a square with a side of 5 mm. The joint top and base layers can be fixed by welding the layers caused by connecting the device to the electrical circuit and pressing. In described embodiment of the invention, for the purpose of rapid plasticizing of the device according to the invention, measuring 35 cm×25 cm, it is placed on a thermal insulator in the form of a textile fabric and then a direct current of 24 V and a current of approximately 4 A is connected The person skilled in the art as part of their routine operation is able to adjust the parameters of the current parameters depending on the size and properties of the sheet. After about 1 minute, the sheet according to the example described becomes plasticized. The device thus prepared matches the shape of the immobilized hand (as shown in FIG. 4). The material typically stiffens after about 1 minute and reaches full stiffness after about 5 minutes. This parameter depends on many variables, including ambient temperature. In order to correct the mapped shape of the device, the device can be reconnected to electrical circuit, for example to a current of 5V and about 2 A, which results in a slow heating of the sheet. It is also possible to heat up the system faster by using a higher voltage, e.g. 24V, but slower heating results in less discomfort. After obtaining sufficient plasticizing of the plastic, the mapped shape of the device is corrected. In the embodiment according to FIG. 4, the opposite ends of the sheet were wrapped around the forearm and joined together by arranging them in a shape of the linen seam. This joint is particularly advantageous because it allows for a perfect fit to the spatial form while maintaining very strong fastening and high aesthetic qualities.

A particular example of a solution according to the invention is a sheet in the form of a mesh formed by the filaments of conductors coated with an insulator in the form of a thermoplastic polymer or a suitable thermoplastic blend based on a flexible polymer, where the mesh elements can optionally be joined together at nodal points. The connection of these elements can be caused by the use of a flexible and conductive heat binder. Alternatively, the filaments may be joined together in the form of knots of welded polymer layers or a polymer-based blend. The eyehole in the mesh can have in any shape, especially can be a square, a rectangle or a hexagon. The ends of the mesh can be connected with a conductor that is not part of the system, and then the system can be connected to a power source in the form of a dedicated device driver.

A conductor for use in a system adapted to generate heat can be, among others, properly insulated: resistance wire, copper wire as well as carbon fiber. It is particularly advantageous to use carbon fiber because it is not visible in image methods using X-rays. This is particularly advantageous when using the device for medical purposes, such as immobilizing a broken limb or in the field of teleradiotherapy, as it allows for performing control tests without disturbing the image caused by the use of the immobilizing dressing such as the scales.

It is also possible to provide a device consisting of areas that can be heated and, consequently, plasticized, independently from one another, which can be used when repositioning parts of the device without need to plasticize the entire device.

The heating process takes place much faster when the device is placed on a thermo-insulator, thanks to which heat losses to the environment are minimized.

The use of sheets with thermoplastic materials and a system adapted to generate plasticizing heat for the sheet, makes it possible to precisely adjust the shape according to the user's intentions without the time constraints due to the hardening of the sheet. The possibility of reheating the system allows for plasticizing the material and possible corrections in order to set it properly also in the case where hardening of the plastic occurs before the end of forming the target shape, for example to ensure adequate comfort for the user (patient), or in case of later correction of immobilized object. The solution according to the invention allows multiple shape adjustments if the mold without any time constraints associated with the time of material stiffening, as well as reuse of the device due to the possibility of regenerating its shape to a sufficient extent for its subsequent use. Thanks to the possibility of matching the sheet to the size and shape of the expected spatial form, it is possible to use it conveniently for many applications of mapping the shape of the spatial forms, objects such as, for example, anatomical parts of the body or sculptures. In contrast to the solutions available on the market and based on sheets made using thermoplastic materials, in use of the solution according to the invention there is no need to place the device in the area of elevated temperature every time, and therefore there is no need to employ additional large-size devices. In medical applications, the solution allows, for example, to adjust fractures with a stiffening bandage already installed or a reposition of improperly set bone fragments.

1. A device for mapping the shape of a spatial form comprising a thermoplastic sheet, the thermoplastic sheet is provided with a flexible system for generating heat to plasticize said sheet due to the flow of current,
characterized in that
said system for generating heat is provided in the form of a system or systems of conductor arranged in a thermoplastic sheet to form the shape of a mesh and in that perforation is provided in the areas of the sheet defined between the conductors forming the system for generating heat.

2. The device according to claim 1, characterized in that the system for generating heat is equipped with current connecting means.

3. The device according to claim 1, characterized in that the thermoplastic sheet consists of a top layer and a base layer, wherein on the surface of the base layer facing the top layer grooves are provided for receiving conductor laid in the layer.

4. The device according to claim 3, characterized in that on the surface of the top layer facing the base layer there are provided projections in a size and shape substantially corresponding to the size and shape of the groves in the base layer, wherein the height of said projections is smaller than the depth of the groves, and the difference between the groove depth and the projection height substantially corresponds to the height of the conductor or conductors laid in the base layer.

5. The device according to claim 4, characterized in that said layers are joint with a flexible and heat conducting binder.

6. The device according to claim 5, characterized in that the binder is a universal silicone.

7. The device according to claim 3, characterized in that said layers are made by injection molding or by use a 3D printer or by use of stamped molds.

8. The device according to claim 1, characterized in that the thermoplastic sheet is made of a material selected from thermoplastic polymers, in particular thermoplastic elastomers such as thermoplastic polyurethanes, thermoplastic polyisoprenes, thermoplastic polyesters, thermoplastic polyolefins, polyvinylchloride, polystyrene, blends of two or more of these materials.

9. The device according to claim 1, characterized in that the thermoplastic sheet is made of thermoplastic polyolefins selected from the group consisting of isotactic polypropylene, ethylene 1-butene copolymers, ethylene 1-ethene copolymer; poly-&-caprolactone; polycaprolactone-containing thermoplastic polyurethane or blend of two or more of these materials.

10. The device according to claim 1, characterized in that the thermoplastic sheet is made of a blend based on polycaprolactone with the addition of plasticizers.

11. The device according to claim 1, characterized in that the thermoplastic sheet is made of a material having a softening point in the range of 38 to 100 degrees Celsius.

12. The device according to claim 1, characterized in that it comprises means for connecting, preferably detachable connecting, the opposite edges of the thermoplastic sheet.

13. The device according to claim 1, characterized in that it comprises a heat-insulating layer on the surface of a thermoplastic sheet.

14. The device according to claim 1, wherein the thermoplastic sheet under current flow immobilize a human or animal body part, in particular a limb or a joint.

15. The device according to claim 1, further comprises a controller for controlling the parameters of the system for generating heat.
